(19) United States
(12) Patent Application Publication
(10) Pub. No.: US 2023/0317209 A1
(43) Pub. Date: Oct. 5, 2023

(54) METHOD FOR SCREENING SPLIT SITES AND APPLICATION THEREOF

(71) Applicant: Shenzhen Haolthy Biotechnology Co., Ltd., Shenzhen (CN)

(72) Inventor: Hao WANG, Shenzhen (CN)

(21) Appl. No.: 17/844,338

(22) Filed: Jun. 20, 2022

(30) Foreign Application Priority Data
Mar. 31, 2022 (CN) 202210331773.2

(51) Int. Cl. G16B 35/20; G16B 20/00; G16B 50/30; G16B 15/00; G16B 40/00; G16B 35/10; G01N 33/68

(52) U.S. Cl. CPC G16B 35/20; G16B 20/00; G16B 50/30; G16B 15/00; G16B 40/00; G16B 35/10; G01N 33/6848

(57) ABSTRACT

A method for screening a split site and an application thereof are provided. The method includes: S1, writing a program using a computer language, and predicting an amino acid sequence formed by connecting adjacent peptide fragments after an intein is embedded into each two adjacent amino acid residues in an initial amino acid sequence and then excised through a self-splicing reaction to construct a protein database; and S2, performing molecular clone after inserting an intein sequence into a gene segment and then translating to obtain a peptide fragment, detecting whether that peptide fragment contain a labeled amino acid sequence by mass spectrometry, and comparing the peptide fragment with the protein database to confirm the split site. A final detection is realized by the mass spectrometry instead of high-throughput screening, and extended to searches for the split site of any active protein.

### TECHNICAL FIELD

The disclosure relates to fields of protein split site screening technologies, more particular to a method for screening split sites and an application thereof.

### SEQUENCE LISTING

This application incorporates by reference the material in the sequence listing submitted via ASCII text file titled sl.txt, with the data of creation being Jun. 20, 2022, and the size of the ASCII text file being 4 KB.

### BACKGROUND

Protein introns (also referred to as inteins) are able to connect flanking external proteins (also referred to as exteins) into a new protein fragment and excise themselves, and this process is called protein splicing. Inteins are found in many natural organisms, such as bacteria, fungi and lower plants, often embedded in important proteins. In nature, a protein splicing produces two separate proteins (intein and extein) and under the control of a gene, the intein precisely excises an internal protein fragment (i.e., the intein itself) and connects two regions at the same time. There are several forms of inteins in nature, including full-length inteins, mini inteins and naturally occurring split inteins. Both the full-length inteins and the mini inteins are cis-splicing inteins with or without an endonuclease domain. Split inteins are trans-splicing inteins that contain two protein fragments and are transcribed and translated from two separate genes. Trans-splicing requires co-expression of two slit intein fragments, namely, an N-terminal intein fragment (IN, fused with a C-terminal of N-extein) and a C-terminal intein fragment (IC, fused with a N-terminal of C-extein). The split intein fragments then bind to restore its activity and catalyzes a connection of the N-extein and the C-extein. In the cis-splicing or the trans-splicing, an intein-mediated splicing reaction does not require a help of any enzyme or cofactor (also referred to as helper factors), but only requires that expressed proteins have correct folding structures.

The existing scientific literature (Ho, T. Y. H., Shao, A., Lu, Z. et al., “A systematic approach to inserting split inteins for Boolean logic gate engineering and basic activity reduction”, NATURE COMMUNICATIONS, 2021, Nat Commun 12, 2200) records a method for screening protein split sites, entitled Intein-assisted bisection mapping (IBM) split site screening. It is also a method of random insertion of gene segments by mini-Mu transposon mediated by phage infection mechanism, but it is only limited to some fluorescent proteins or antibiotic resistance proteins that have been successfully searched for split sites. These proteins themselves can stimulate fluorescence or play a regulatory role in inhibiting antibiotics and transcriptional promoters, making them have characteristics of activity that can be detected by high-throughput screening. However, most recombinant proteins, such as interleukin 2 (IL-2), interferon (IFN), epidermal growth factor (EGF), basic fibroblast growth factor (bFGF) and other high-value protein medicines, do not have the characteristics that can be directly detected by the high-throughput screening.

### SUMMARY

In view of the above problems existing in the related art, a purpose of the disclosure is to provide a method for screening split sites. A protein database is constructed through computer programming, and then experiments are performed to detect and verify the split sites through mass spectrometry. A final detection is realized by the mass spectrometry instead of high-throughput screening, which can be extended to search for the split sites of any active protein.

As a premise, an experimental principle of the disclosure (i.e., a method of random insertion of gene segments by mini-Mu transfer mediated by phage infection mechanism) is the same as the above-mentioned scientific literature “A systematic approach to inserting split intents for Boolean logic gate engineering and basic activity reduction”, which may also be used as an explanation if expressions of the relevant experimental principle are not clear enough in the text of the disclosure.

To achieve the above purpose, a first aspect of the disclosure provides a method for screening a split site, including:

step S1, establishing a protein database, which includes: writing a program by using a computer language, and predicting an amino acid sequence formed by connecting adjacent peptide fragments after an intein is embedded into each two adjacent amino acid residues in an initial amino acid sequence and then excised through a self-splicing reaction to construct the protein database; and

step S2, performing an experiment, which includes: inserting an intein sequence into a gene segment through a molecular clone experimental method and then translating to obtain a peptide fragment, detecting whether that peptide fragment contain a labeled amino acid sequence by mass spectrometry, and comparing the peptide fragment with the protein database when the peptide fragment is detected as containing the labeled amino acid sequence to confirm the split site.

In the disclosure, the computer language may employ any language that can realize a programming function, such as writing scripts in Python to complete the establishment of the protein database in the step S1 of the disclosure.

A principle that the mass spectrometry can be used for detection in the disclosure is described as follows. An enzyme cutting site (also referred to as a restriction enzyme cutting site or a restriction site) left by molecular cloning may also be translated into amino acids during expression, so that whether a labeled amino acid sequence translated by a marker sequence is generated can be used as a sign of whether the splicing reaction occurs. By detecting whether the labeled amino acid sequence is contained in the translated peptide through the mass spectrometry, the searches of the split sites of any active protein can be realized, rather than limited to some fluorescent proteins or antibiotic resistance proteins that have successfully searched the split sites.

In some embodiments of the disclosure, the establishing a protein database in the step S1 may specifically include:

step S11, fusing a first gene segment (also referred to as gene segment 1, including SEQ ID NO: 6 and SEQ ID NO: 7), an inserted intein sequence segment, and a second gene segment (also referred to as gene segment 2, including SEQ ID NO: 7, SEQ ID NO: 8, and SEQ ID NO: 9) in a sequential order to obtain a new DNA sequence;

step S12, translating the new DNA sequence into a new amino acid sequence;

step S13, searching a target intein amino acid sequence in the new amino acid sequence (the program sets that the new amino acid sequence contains the target intein amino acid sequence), and deleting the target intein amino acid sequence in the new amino acid sequence to thereby obtain an output amino acid sequence; and

step S14, predicting each possible site of the first gene segment and the second gene segment into which the inserted intein sequence segment is inserted, and repeating the steps S11 to S13 to obtain all the output amino acid sequences to construct the protein data database.

According to the disclosure, in the step S13, the computer programming is set to automatically delete the target intein amino acid sequence in the new amino acid sequence, so as to obtain the output amino acid sequence.

FIG. 1 is a schematic flowchart of database establishment through computer programming according to some embodiments of the disclosure. Referring to FIG. 1. M86 is intein Ssp DnaBM86, which is modified from intein Ssp DnaB found in Synechocystis spp. PCC6803.

In some embodiments of the disclosure, in the step S11, several bases are inserted into the new DNA sequence to prevent frameshift mutation during translation. After phage transposition, the first several (e.g., 5) bases inserted at a 5′-terminal are copied once. Therefore, in order to prevent the frameshift mutation, several (e.g., 1) bases are inserted at a 3′-terminal of the copied 5 bases (the 5′-terminal of the inserted intein sequence segment) in the inserted intein sequence segment, so that a sum of the number of the copied bases after the phage transposition and the number of bases actively inserted meets a multiple of 3. FIG. 1 is a schematic diagram of the inserted segment in some embodiments of the disclosure. Referring to FIG. 2, five bases (SEQ ID NO: 7) from i-5 to i are the first five bases duplicated during the transposition. After inserting intein gene segment whose 5′-terminal and 3′-terminal are respectively provided with enzyme cutting sites and added with several bases, the five bases are added at a downstream thereof, the whole gene segment are translated into an amino acid sequence, and then the intein sequence is deleted to obtain one of amino acid sequences in the protein database. The above steps are compiled into a computer program language (such as a Python program) to insert and deduce the intein between each adjacent two amino acids in a target protein to obtain the finally constructed protein database (referring to the description of the step S1).

According to the disclosure, the mini-Mu transposition mechanism duplicates five bases at an upstream of an insertion position once at a downstream of the insertion position, thus the marker sequence finally left by a combination of the restriction enzyme cutting site and the transposition mechanism

A second aspect of the disclosure provides a use of the method described in the first aspect in screening of split sites of at least one of Escherichia coli (E. coli) antigen protein Im7-6 and Cas9 protein, where Im7-6 refers to immunity protein 7-6, and Cas9 refers to clustered regularly interspaced short palindromic repeats associated protein 9.

A third aspect of the disclosure provides a use of the method described in the first aspect in screening recombinant proteins such as interleukin 2 (IL-2), interferon (IFN), epidermal growth factor (EGF), and basic fibroblast growth factor (bFGF).

Since high-throughput screening is used in the above scientific literature, it is limited to some fluorescent proteins or antibiotic resistance proteins that have successfully searched for split sites, and these proteins themselves can excite fluorescence or play a regulatory role in inhibiting antibiotics and transcription initiation, so that they have characteristics of activity that can be detected by high-throughput screening The disclosure realizes the final detection through mass spectrometry, and expands it to search for the split sites of any active protein.

Beneficial effects of the disclosure may include at least one of the following:

1) the screening method provided by the disclosure may reasonably match with the computer programming to construct the protein database;

2) the screening method provided by the disclosure may realize the final detection through mass spectrometry, innovatively expands the existing screening scheme for the split sites, and can be extended to search for the split sites of any active protein; and

3) after the screening method provided by the disclosure has confirmed the split sites, the subsequent experiment can be designed for protein assembly, providing a new idea for the subsequent glycosylation experiment.

### BRIEF DESCRIPTION OF DRAWINGS

FIG. 1 illustrates a schematic flowchart of database establishment through computer programming according to some embodiments of the disclosure.

FIG. 2 illustrates a schematic diagram of inserting a segment according to some embodiments of the disclosure.

FIG. 3 illustrates a schematic diagram of operation steps of randomly inserting gene segments according to a first embodiment of the disclosure.

FIG. 4 illustrates a schematic diagram of a nucleotide sequence of intein Ssp DnaBM86 according to the first embodiment of the disclosure.

FIG. 5 illustrates a schematic diagram of a screening method according to the first embodiment of the disclosure.

FIG. 6 illustrates a result of polypeptide split sites detected by mass spectrometry according to the first embodiment of the disclosure.

FIG. 7 illustrates a schematic diagram of a nucleotide sequence of a split intein Ssp DnaBM86 according to a second embodiment of the disclosure.

FIG. 8 illustrates a first result of polypeptide split sites detected by mass spectrometry according to the second embodiment of the disclosure.

FIG. 9 illustrates a second result of polypeptide split sites detected by mass spectrometry according to the second embodiment of the disclosure.

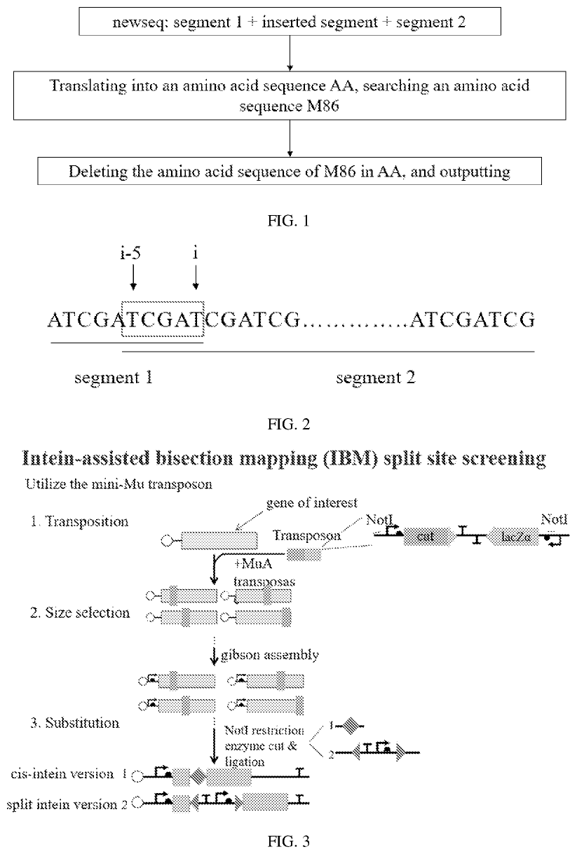

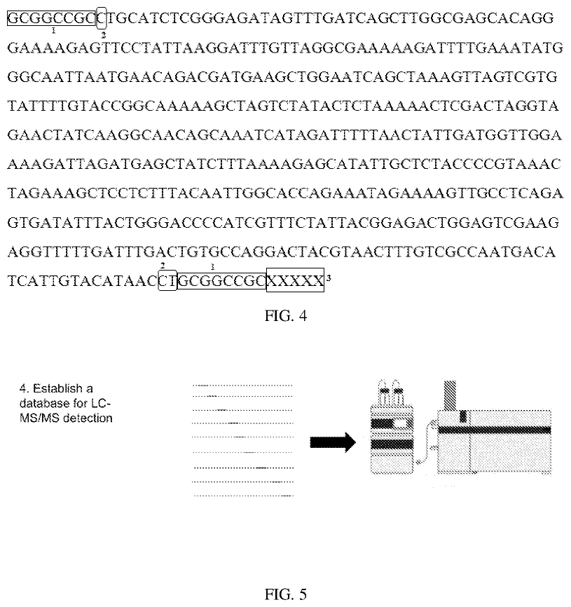

### DETAILED DESCRIPTION OF EMBODIMENTS

In order that the disclosure may be easily understood, the disclosure will be described in detail below in combination with the accompanying drawings. However, before describing the disclosure in detail, it should be understood that the disclosure is not limited to specific embodiments described. It should also be understood that terms used herein are for the purpose of describing specific embodiments only and are not intended to be limiting.

When a value range is provided, it should be understood that upper and lower limits of the range and each intermediate value between any other specified or intermediate values in the range are included in the disclosure. The upper and lower limits of these smaller ranges may be independently included in the respective smaller ranges, and are also included in the disclosure, subject to any explicitly excluded limits in the specified ranges. Where the specified range includes one or two limits, a range excluding any or both of those included limits is also included in the disclosure.

Unless otherwise defined, all terms used herein have the same meaning as commonly understood by those skilled in the art to which the disclosure belongs. While any methods and materials similar or equivalent to those described herein can also be used in implementation or testing of the disclosure, preferred methods and materials are described herein.

The relevant experimental principle of the method for randomly inserting gene segments by mini-Mu transposon mediated by phage infection mechanism adopted by the disclosure can be referred to a scientific and technical literature (Ho, T. Y. H., Shao, A., Lu, Z. et al., “A systematic approach to inserting split inteins for Boolean logic gate engineering and basic activity reduction”, NATURE COMMUNICATIONS, 2021, Nat Commun 12, 2200), and the full text of the literature may be cited for reference.

### First Embodiment

Referring to FIG. 3 and FIG. 4, in this embodiment, the above screening method is used to verify polypeptide split site of Escherichia coli (E. coli) antigen proteins (e.g., immunity protein 7-6 (Im7-6)).

As shown in FIG. 3, FIG. 3 has the same experimental principle as the above scientific literature “A systematic approach to inserting split inteins for Boolean logic gate engineering and basic activity reduction”, that is, the method for randomly inserting gene segments by mini-Mu transposon mediated by phage transposition mechanism. The specific operations are as follows.

1. Transposase MuA is used to perform transposition experiment on a target gene segment, transposon is randomly inserted into the target gene segment, and its principle ensured that only one transposon segment is inserted into each the target gene segment. The transposon segment is a complete expression line with a promoter, a terminator and other elements and expressed chloramphenicol resistance protein.

2. The transposed gene segment is connected to pET28a expression vector by seamless cloning method and transformed into E. coli Top10 amplification vector. The colonies are screened by a chloramphenicol resistant plate.

3. All the colonies in the above plate are collected, mixed and cultured, and their plasmids are extracted. Since the transposon carriers a NotI restriction enzyme cutting site at each upstream and downstream terminals, the transposon segment is replaced by the expressed intein Ssp DnaBM86 (corresponding to a cis-intein version of 3. substitution in FIG. 3), and then transformed into competent E. coli Top10 for vector reproduction. Kanamycin antibiotic plate is used for colony screening. Then, all colonies are collected and cultured, the plasmids are extracted, transformed them into E. coli BL21DE3 expression strain, and all the colonies are collected again and then inoculated into Lysogeny broth (LB) medium for protein expression. Finally, the mixed expressed proteins are purified and concentrated to obtain samples.

FIG. 4 illustrates a schematic diagram of a nucleotide sequence of intein Ssp DnaBM86 with restriction enzyme cutting sites and additional bases added to prevent frameshift mutation. In the FIG. 4, where showing SEQ ID NO: 1, GCGGCCGC in two boxes 1 is a nucleotide sequence of the restriction enzyme cutting site, base C in the first box 2 and CT bases in the second box 2 are additional bases added to prevent the frameshift mutation, and XXXXX (X represents a base selected from bases A, T, C, and G) in box 3 is five bases duplicated after phage transposition. It should be noted that FIG. 4 shows only part of the nucleotide sequence, especially the five bases copied in box 3 are not fixed.

As shown in FIG. 5, the disclosure is significantly different from the above-mentioned scientific and technical literature (the literature directly conducts high-throughput screening on samples). The specific operations are as follows:

4. A script is written first by using Python to predict that the intein has undergone a splicing reaction to excise itself after being embedded in each of the two adjacent amino acid residues in the amino acid sequence, and the amino acid sequence formed after the adjacent peptide fragments are connected would form a protein database.

5. The samples obtained in the step 3 is detected by mass spectrometry.

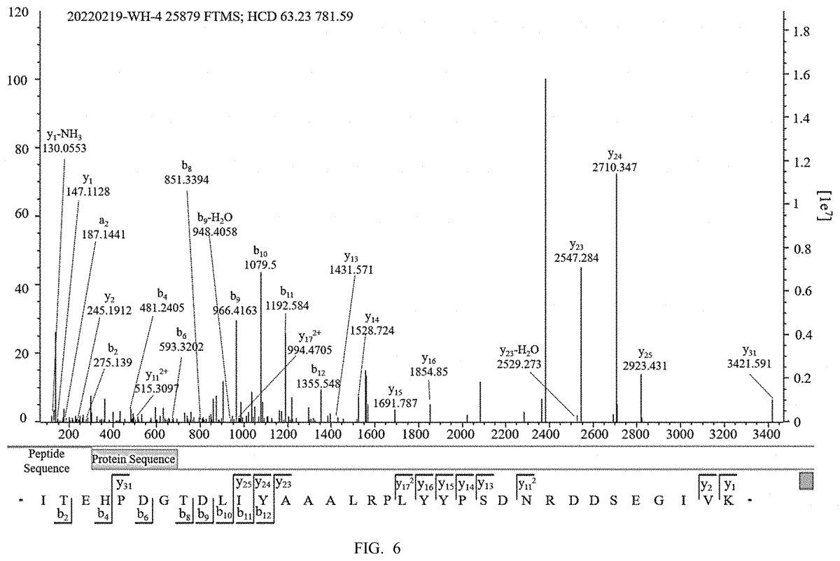

A result of mass spectrometry is shown in FIG. 6. From the analysis in FIG. 6, for E. coli antigen protein (e.g., Im7-6), an amino acid sequence of AAALRPLY (SEQ ID NO: 2) is a labeled amino acid sequence translated from the marker sequence generated at the restriction enzyme cutting site (the mini-Mu transposition mechanism duplicates five bases at the upstream of the insertion position once at the downstream of the insertion position, thus the marker sequence left by the combination of the restriction enzyme cutting sites and the transposition mechanism is a sequence containing eight amino acid residues: AAALRPXX, where XX is translated from the five bases duplicated and one base inserted to prevent the frameshift mutation). Then, the protein database obtained in the step 4 is searched, and when an ion fragment fully or partially covering (partially covering here means that at least two amino acids A, and L are found to prove that the splicing reaction of the intein actually takes place) the marker sequence is found, it can be determined that a polypeptide split site of E. coli antigen protein (Im7-6) is Y61-Y62 (as shown in FIG. 5). Herein, in SEQ ID NO: 2, A refers to alanine abbreviated as Ala, L refers to leucine abbreviated as Leu, R refers to arginine abbreviated as Arg, P refers to proline abbreviated as Pro, and Y refers to tyrosine abbreviated as Tyr.

### Second Embodiment

In this embodiment, the above screening method is used to verify two polypeptide split sites of clustered regularly interspaced short palindromic repeats (CRISPR) associated protein 9 (Cas9).

Steps 1-5 are substantially the same as those in the first embodiment, except that, in the step 3, “the transposon segment is replaced by the expressed intein Ssp DnaBM86 through enzyme digestion (also referred to as enzyme-cut)” is replaced by “the transposon segment is replaced by the gene segment that an N-terminal of the split intein Ssp DnaBM86, transcriptional ulator elements (TREs) including terminator and promoter, and a C-terminal of the split intein Ssp DnaBM86 (corresponding to a split intein version of 3. substitution in FIG. 3)”.

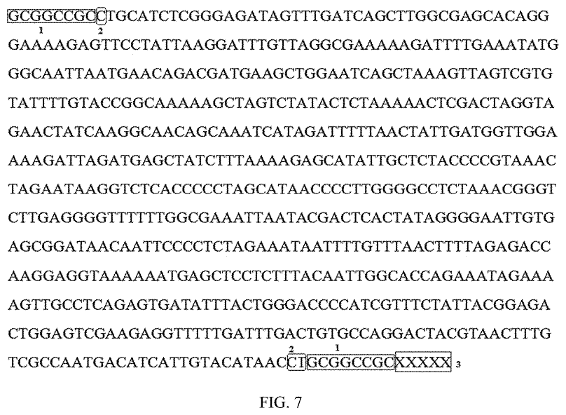

FIG. 7 illustrates a schematic diagram of a nucleotide sequence of the intein Ssp DnaBM86 with restriction enzyme cutting sites and additional bases added to prevent frameshift mutation. As described in FIG. 4 above, in the FIG. 7, where showing SEQ ID NO: 3, GCGGCCGC in two boxes 1 is a nucleotide sequence of a restriction enzyme cutting site, C base in the first box 2 and CT bases in the second box 2 are the additional bases added to prevent the frameshift mutation, and XXXXX (X represents a base selected from bases A, T, C, and G) in box 3 is five bases duplicated after phage transposition. Accordingly, FIG. 7 also shows only part of the nucleotide sequence, especially the five bases copied in box 3 are not fixed.

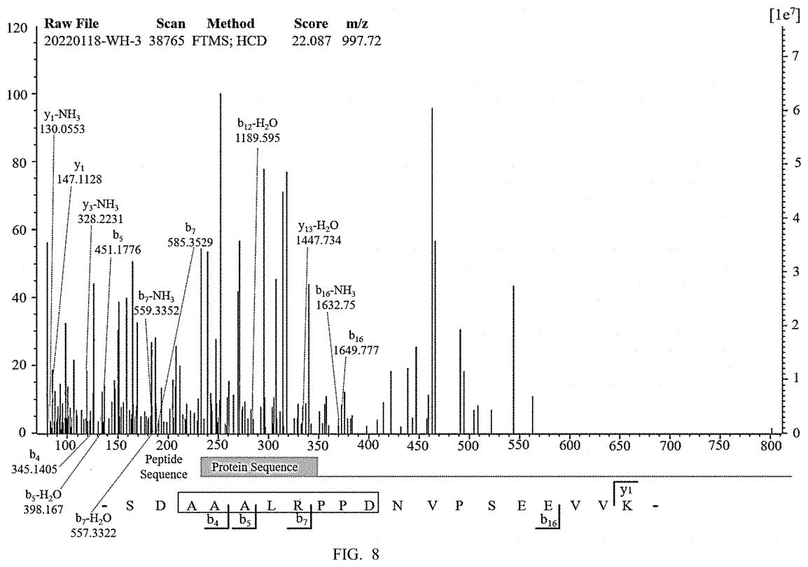

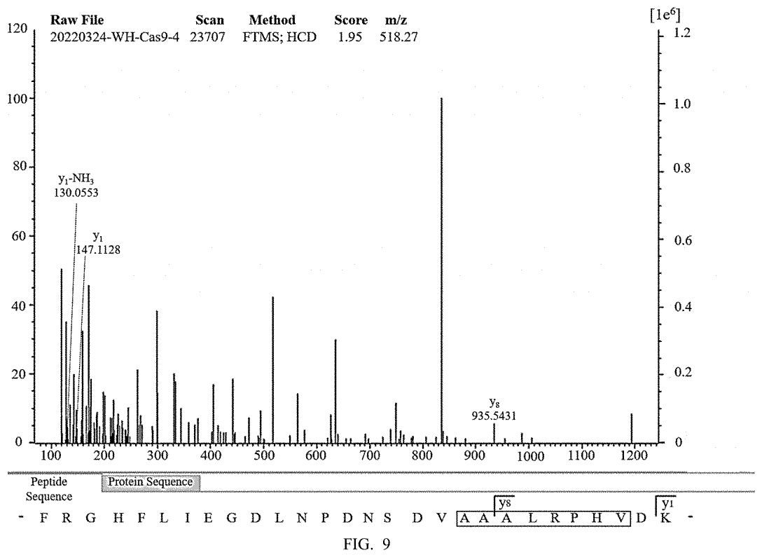

Results of mass spectrometry are shown in FIG. 8 and FIG. 9. From the analysis. for Cas9 protein, amino acid sequences of AAALRPPD (SEQ ID NO: 4) and AAALRPHV (SEQ ID NO: 5) are labeled amino acid sequences translated from the marker sequences generated at the restriction enzyme cutting sites, respectively (the phage Mu transposition mechanism duplicates five bases at the upstream of the insertion position once at the downstream of the insertion position, so the marker sequence left by the combination of the restriction enzyme cutting sites and the transposition mechanism is a sequence containing eight amino acid residues: AAALRPXX, where XX is translated from the five bases duplicated and three bases inserted to prevent the frameshift mutation). Then, the protein database obtained in the step 4 is searched, and when an ion segment fully or partially covering (partially covering here means that at least two amino acids A and L are found to prove that the splicing reaction of the intein actually takes place) the marker sequence is found, it can be determined that two polypeptide split sites of Cas9 protein are D868-N869 (FIG. 6) and 181V-182D (FIG. 7) respectively. Herein, in SEQ ID NO: 4 and SEQ ID NO: 5, D refers to aspartic acid abbreviated as Asp, H refers to histidine abbreviated as His, and V refers to valine abbreviated as Val.

It can be seen from the above embodiments that the screening method provided in the disclosure is reasonably matched with computer programming to construct the protein database; and the final detection is realized by mass spectrometry, which innovatively expands the existing screening scheme for the split sites, and can be extended to search for the split sites of any active protein. After confirming the split sites, experiments can be designed for protein assembly, which provides a new idea for the subsequent glycosylation experiments.

It should be noted that the above-described embodiments are only for the purpose of illustrating the disclosure and do not constitute any limitation of the disclosure. The disclosure has been described with reference to exemplary embodiments, but it should be understood that the words used therein are words of description and explanation, not of limitation. The disclosure may be modified within the scope of the claims of the disclosure, and the disclosure may be modified without departing from the scope and spirit of the disclosure. Although the disclosure described herein relates to specific methods, materials, and embodiments, it does not mean that the disclosure is limited to the specific embodiments disclosed therein. On the contrary, the disclosure may be extended to all other methods and applications with the same function.

What is claimed is:

1. A method for screening a split site, comprising:
step S1, establishing a protein database, which comprises: writing a program by using a computer language, and predicting an amino acid sequence formed by connecting adjacent peptide fragments after an intein is embedded into each two adjacent amino acid residues in an initial amino acid sequence and then excised through a self-splicing reaction to construct the protein database; and
step S2, performing an experiment, which comprises: inserting an intein sequence into a gene segment through a molecular clone experimental method and then translating to obtain a peptide fragment, detecting whether that the peptide fragment contains a labeled amino acid sequence by mass spectrometry, and comparing the peptide fragment with the protein database when the peptide fragment is detected as containing the labeled amino acid sequence to confirm the split site.

2. The method according to claim 1, wherein in the step S1, the establishing a protein database specifically comprises:
step S11, fusing a first gene segment, an inserted intein sequence segment, and a second gene segment in a sequential order to obtain a new deoxyribonucleic acid (DNA) sequence;
step S12, translating the new DNA sequence into a new amino acid sequence;
step S13, searching a target intein amino acid sequence in the new amino acid sequence, and deleting the target intein amino acid sequence in the new amino acid sequence to thereby obtain an output amino acid sequence; and
step S14, predicting each possible site of the first gene segment and the second gene segment into which the inserted intein sequence segment is inserted, and repeating the steps S11 to S13 to obtain all the output amino acid sequences to construct the protein data database.

3. The method according to claim 2, wherein in the step S11, at least one base is inserted into the inserted intein sequence segment.

4. The method according to claim 3, wherein the at least one base is one base.

5. A use of the method according to claim 1 in screening split sites of at least one of Escherichia coli (E. coli) antigen protein Im7-6 and Cas9 protein, wherein Im7-6 refers to immunity protein 7-6, and Cas9 refers to clustered regularly interspaced short palindromic repeats associated protein 9.

6. The use of claim 5, wherein in the step S1, the establishing a protein database specifically comprises:
step S11, fusing a first gene segment, an inserted intein sequence segment, and a second gene segment in a sequential order to obtain a new deoxyribonucleic acid (DNA) sequence;
step S12, translating the new DNA sequence into a new amino acid sequence;
step S13, searching a target intein amino acid sequence in the new amino acid sequence, and deleting the target intein amino acid sequence in the new amino acid sequence to thereby obtain an output amino acid sequence; and
step S14, predicting each possible site of the first gene segment and the second gene segment into which the inserted intein sequence segment is inserted, and repeating the steps S11 to S13 to obtain all the output amino acid sequences to construct the protein data database.

7. The use of claim 6, wherein in the step S11, at least one base is inserted into the inserted intein sequence segment.

8. The use of claim 7, wherein the at least one base is one base.


SEQUENCE LISTING (9 sequences)

SEQ ID NO: 1 - DNA, 481 bp, Artificial Sequence
  Ssp DnaBM86
gcggccgcctgcatctcgggagatagtttgatcagcttggcgagcacagggaaaagagttcctattaaggatttgttaggcgaaaaagattttgaaatatgggcaattaatgaacagacgatgaagctggaatcagctaaagttagtcgtgtattttgtaccggcaaaaagctagtctatactctaaaaactcgactaggtagaactatcaaggcaacagcaaatcatagatttttaactattgatggttggaaaagattagatgagctatctttaaaagagcatattgctctaccccgtaaactagaaagctcctctttacaattggcaccagaaatagaaaagttgcctcagagtgatatttactgggaccccatcgtttctattacggagactggagtcgaagaggtttttgatttgactgtgccaggactacgtaactttgtcgccaatgacatcattgtacataacctgcggccgc

SEQ ID NO: 2 - protein, 8 aa, Artificial Sequence
  Im7-6
AAALRPLY

SEQ ID NO: 3 - DNA, 645 bp, Artificial Sequence
  split intein Ssp DnaBM86
gcggccgcctgcatctcgggagatagtttgatcagcttggcgagcacagggaaaagagttcctattaaggatttgttaggcgaaaaagattttgaaatatgggcaattaatgaacagacgatgaagctggaatcagctaaagttagtcgtgtattttgtaccggcaaaaagctagtctatactctaaaaactcgactaggtagaactatcaaggcaacagcaaatcatagatttttaactattgatggttggaaaagattagatgagctatctttaaaagagcatattgctctaccccgtaaactagaataaggtctcaccccctagcataaccccttggggcctctaaacgggtcttgaggggttttttggcgaaattaatacgactcactataggggaattgtgagcggataacaattcccctctagaaataattttgtttaacttttagagaccaaggaggtaaaaaatgagctcctctttacaattggcaccagaaatagaaaagttgcctcagagtgatatttactgggaccccatcgtttctattacggagactggagtcgaagaggtttttgatttgactgtgccaggactacgtaactttgtcgccaatgacatcattgtacataacctgcggccgc

SEQ ID NO: 4 - protein, 8 aa, Artificial Sequence
  Cas9
AAALRPPD

SEQ ID NO: 5 - protein, 8 aa, Artificial Sequence
  Cas9
AAALRPHV

SEQ ID NO: 6 - DNA, 5 bp, Artificial Sequence
  part of segment 1
atcga

SEQ ID NO: 7 - DNA, 5 bp, Artificial Sequence
  inserted segment
tcgat

SEQ ID NO: 8 - DNA, 6 bp, Artificial Sequence
  part of segment 2
cgatcg

SEQ ID NO: 9 - DNA, 8 bp, Artificial Sequence
  part of segment 2
atcgatcg